(19) United States
(12) Patent Application Publication
(10) Pub. No.: US 2016/0038223 A1
(43) Pub. Date: Feb. 11, 2016

(54) SURGICAL FORCEPS AND METHODS OF MANUFACTURING THE SAME

(71) Applicant: COVIDIEN LP, MANSFIELD, MA (US)

(72) Inventor: GARY M. COUTURE, WARD, CO (US)

(21) Appl. No.: 14/794,029

(22) Filed: Jul. 8, 2015

Related U.S. Application Data
(60) Provisional application No. 62/035,747, filed on Aug. 11, 2014.
(60) Provisional application No. 62/035,737, filed on Aug. 11, 2014.

(51) Int. Cl. A61B 18/14; A61B 17/285; A61B 17/29

(52) U.S. Cl. CPC A61B 18/1445; A61B 17/2909; A61B 17/285; A61B 18/1447; A61B 2017/2926; A61B 2018/1455

(57) ABSTRACT

A forceps includes an integral member having a body portion, first and second handles, and a connector member. The body portion defines a lumen extending longitudinally therethrough. The first and second handles are integrally formed with the body portion via living hinges and are movable relative to one another between a spaced-apart position and an approximated position. The connector member is integrally formed between the first and second handles and includes a first leg coupled to the first handle via a living hinge, a second leg coupled to the second handle via a living hinge, and a hub coupled between the first and second legs via living hinges. Movement of the first and second handles between the spaced-apart and approximated positions urges the hub to translate relative to the body portion. The integral member is adapted to receive a shaft that supports an end effector assembly for treating tissue.

### CROSS-REFERENCE TO RELATED APPLICATIONS

This application claims the benefit of, and priority to, U.S. Provisional Patent Application Nos. 62/035,747 and 62/035,737, both of which were filed on Aug. 11, 2014. This application is related to U.S. Patent Application No. __/___,___, filed on _______. The entire contents of each of the above applications are hereby incorporated herein by reference.

### BACKGROUND

1. Technical Field

The present disclosure relates to surgical instruments and, more particularly, to surgical forceps configured for treating and/or cutting tissue, and methods of manufacturing the same.

2. Background of Related Art

A surgical forceps is a plier-like device which relies on mechanical action between its jaws to grasp, clamp, and constrict tissue. Energy-based surgical forceps utilize both mechanical clamping action and energy to affect hemostasis by heating tissue to coagulate and/or cauterize tissue. Certain surgical procedures require more than simply cauterizing tissue and rely on the unique combination of clamping pressure, precise energy control, and gap distance (i.e., the distance between opposing jaw members when closed about tissue) to “seal” tissue. Typically, once tissue is treated, the surgeon has to accurately sever the tissue along the newly formed tissue seal. Accordingly, surgical forceps have been designed which incorporate a knife or blade member which effectively severs the tissue after the tissue has been treated.

Generally, surgical instruments, including forceps, can be classified as disposable instruments, e.g., instruments that are discarded after a single use, or reusable instruments, e.g., instruments capable of being sterilized for repeated use. As can be appreciated, those instruments that are configured for single-use must be cost-efficient while still being capable of effectively performing their intended functions.

### SUMMARY

As used herein, the term “distal” refers to the portion that is being described which is further from a user, while the term “proximal” refers to the portion that is being described which is closer to a user. Further, to the extent consistent, any of the aspects described herein may be used in conjunction with any or all of the other aspects described herein.

In accordance with aspects of the present disclosure, a forceps is provided. The forceps includes an integral member having a body portion defining a lumen extending longitudinally therethrough, first and second handles integrally formed with the body portion, and a connector member integrally formed between the handles. The first and second handles are coupled to the body portion via living hinges and are movable relative to one another between a spaced-apart position and an approximated position. The connector member is coupled between the first and second handles and includes a first leg coupled to the first handle via a living hinge, a second leg coupled to the second handle via a living hinge, and a hub coupled between the first and second legs via living hinges. As a result of this configuration, movement of the first and second handles between the spaced-apart position and the approximated position urges the hub to translate relative to the body portion. The integral member is adapted to receive a shaft that support an end effector assembly for treating tissue.

In an aspect of the present disclosure, the hub of the integral member is configured to engage a drive assembly disposed within the shaft such that movement of the first and second handles to the approximated position urges the hub to translate the drive assembly to actuate the end effector assembly.

In another aspect of the present disclosure, the end effector assembly includes first and second jaw members movable from an open position to a closed position for grasping tissue. The drive assembly, in such aspects, may include a drive bar configured such that movement of the first and second handles from the spaced-apart position to the approximated position urges the hub to translate distally relative to the body portion, thereby translating the drive bar of the drive assembly distally relative to the end effector assembly to move the first and second jaw members from the open position to the closed position.

In another aspect of the present disclosure, the drive assembly includes a mandrel disposed about the proximal end of the drive bar. The mandrel is engaged within a lumen defined through the hub.

In still another aspect of the present disclosure, one or both of the jaw members is adapted to connect to a source of energy for conducting energy between the jaw members to treat tissue grasped therebetween.

In yet another aspect of the present disclosure, a knife is slidably disposed within the drive bar. The knife is movable between a retracted position, wherein the knife is positioned proximally of the end effector assembly, and an extended position, wherein the knife extends at least partially between the jaw members to cut tissue grasped therebetween.

In still yet another aspect of the present disclosure, a trigger assembly including a trigger and a trigger drive bar is provided. The trigger drive bar defines a proximal end and a distal end. The knife is engaged to the distal end of trigger drive bar while the trigger is coupled to the proximal end of the trigger drive bar. The trigger is movable between a proximal position and a distal position for moving the knife between the retracted position and the extended position.

In another aspect of the present disclosure, the trigger is positioned adjacent a cut-out defined within the body portion of the integral member and the trigger drive bar extends through the drive bar of the drive assembly.

In yet another aspect of the present disclosure, the integral member includes a leaf spring integrally formed therewith and extending into the cut-out. The leaf spring is positioned to bias the trigger proximally, thereby biasing the knife towards the retracted position.

In still another aspect of the present disclosure, the trigger assembly includes a post interconnecting the trigger and the trigger drive bar. In such aspects, the leaf spring may include an engagement member disposed at a free end thereof that is configured to at least partially receive the post in operable engagement therewith to bias the trigger proximally.

Another forceps provided in accordance with aspects of the present disclosure includes an integral member having a body portion defining a lumen extending longitudinally therethrough, first and second handles, a connector member, and a leaf spring. The handles are integrally formed with the body portion via living hinges and are movable relative to one another between a spaced-apart position and an approximated position. The connector member is integrally formed between the handles and includes a first leg coupled to the first handle via a living hinge, a second leg coupled to the second handle via a living hinge, and a hub coupled between the first and second legs via living hinges. Movement of the first and second handles between the spaced-apart position and the approximated position urges the hub to translate relative to the body portion. A leaf spring is alsp integrally formed with the body portion. The integral member is adapted to receive a shaft that supports an end effector assembly for treating tissue. The leaf spring is adapted to bias a trigger assembly for actuating a knife relative to the end effector assembly.

In an aspect of the present disclosure, the hub of the integral member is configured to engage a drive assembly disposed within the shaft such that movement of the first and second handles to the approximated position urges the hub to translate the drive assembly to actuate the end effector assembly.

In another aspect of the present disclosure, the end effector assembly includes first and second jaw members movable from an open position to a closed position for grasping tissue. The drive assembly, in such aspects, may include a drive bar configured such that movement of the first and second handles from the spaced-apart position to the approximated position urges the hub to translate distally relative to the body portion, thereby translating the drive bar of the drive assembly distally relative to the end effector assembly to move the first and second jaw members from the open position to the closed position.

In another aspect of the present disclosure, the drive assembly includes a mandrel disposed about the proximal end of the drive bar. The mandrel is engaged within a lumen defined through the hub.

In still another aspect of the present disclosure, one or both of the jaw members is adapted to connect to a source of energy for conducting energy between the jaw members to treat tissue grasped therebetween.

In yet another aspect of the present disclosure, the knife is slidably disposed within the drive bar. The knife is movable between a retracted position, wherein the knife is positioned proximally of the end effector assembly, and an extended position, wherein the knife extends at least partially between the jaw members to cut tissue grasped therebetween.

In still yet another aspect of the present disclosure, the trigger assembly includes a trigger and a trigger drive bar is provided. The trigger drive bar defines a proximal end and a distal end. The knife is engaged to the distal end of trigger drive bar while the trigger is coupled to the proximal end of the trigger drive bar. The trigger is movable between a proximal position and a distal position for moving the knife between the retracted position and the extended position.

In another aspect of the present disclosure, the trigger is positioned adjacent a cut-out defined within the body portion of the integral member and the trigger drive bar extends through the drive bar of the drive assembly.

In yet another aspect of the present disclosure, the leaf spring extends into the cut-out and is positioned to bias the trigger proximally, thereby biasing the knife towards the retracted position.

In still another aspect of the present disclosure, the trigger assembly includes a post interconnecting the trigger and the trigger drive bar. In such aspects, the leaf spring may include an engagement member disposed at a free end thereof that is configured to at least partially receive the post in operable engagement therewith to bias the trigger proximally.

### BRIEF DESCRIPTION OF THE DRAWINGS

Various aspects and features of the present disclosure described herein with reference to the drawings wherein:

FIG. 1 is a top, perspective view of a surgical forceps provided in accordance with the present disclosure;

FIG. 2 is a top, perspective view of the proximal end of the handle member of the forceps of FIG. 1; and

FIG. 3 is an exploded, perspective view of the end effector assembly and drive components of the forceps of FIG. 1.

### DETAILED DESCRIPTION

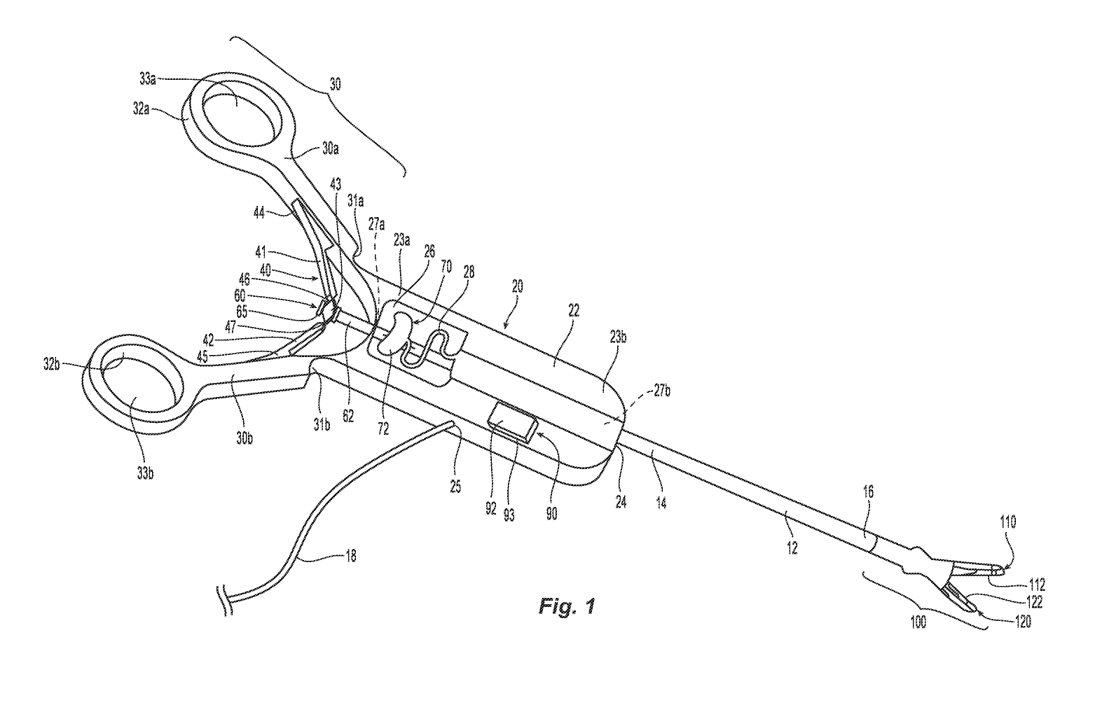

Referring to FIG. 1, an embodiment of a surgical forceps provided in accordance with the present disclosure is shown generally identified by reference numeral 10. Although surgical forceps 10 is shown configured for use in connection with endoscopic surgical procedures, the present disclosure is equally applicable for use in more traditional open surgical procedures and with any suitable surgical instrument.

Forceps 10 is adapted for use in various surgical procedures and generally includes an integral member 20 having a distal body portion 22 and a proximal handle portion 30, a trigger assembly 70, an activation assembly 90, and an end effector assembly 100 which mutually cooperate to grasp, treat, and/or cut tissue. Forceps 10 further includes a shaft 12 having a distal end 16 that mechanically engages end effector assembly 100 and a proximal end 14 that mechanically engages integral member 20. More specifically, proximal end 14 of shaft 12 may be secured within a lumen 24 of integral member 20 via friction fitting, adhesives, or other suitable process. Alternatively, shaft 12 may be integrally formed with integral member 20. A cable 18 is adapted to connect forceps 10 to a source of energy, e.g., a generator (not shown), although forceps 10 may alternatively be configured as a battery powered instrument.

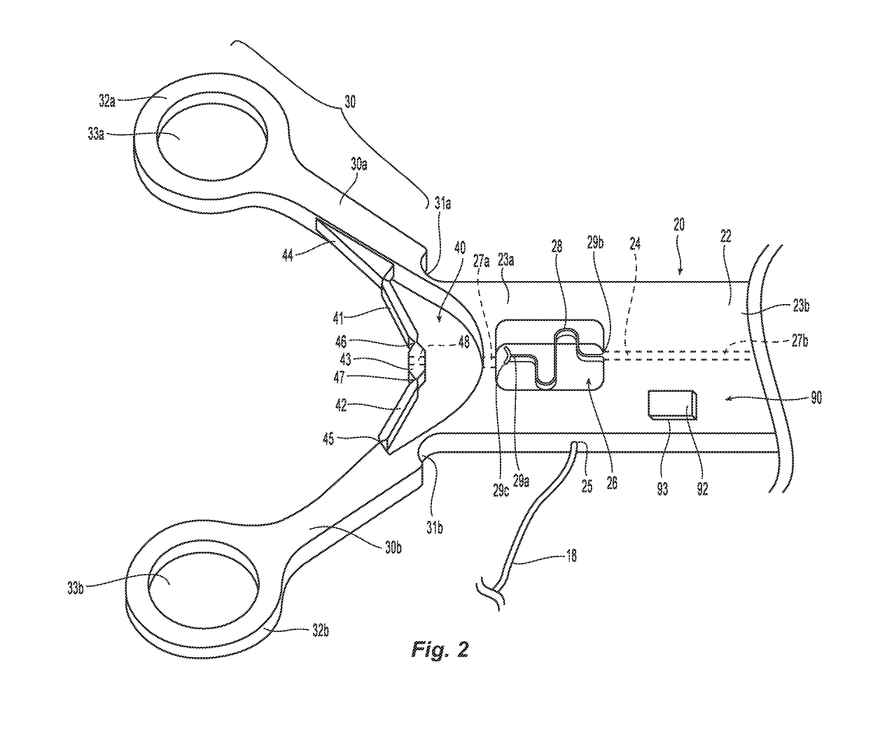

With additional reference to FIG. 2, integral member 20 is monolithically formed via single-shot overmolding, injection molding, or other suitable process and, as mentioned above, includes a distal body portion 22 and a proximal handle portion 30. Distal body portion 22 defines a proximal end 23a, a distal end 23b, and a lumen 24 extending longitudinally through distal body portion 22 from proximal end 23a to distal end 23b thereof. Distal body portion 22 also supports an activation button 92 of actuation assembly 90 and includes an input 25 configured to permit passage of cable 18 into the interior thereof. Cable 18 houses a plurality of wires (not shown) that interconnect the source of energy (not shown) with activation button 92 and end effector assembly 100 to enable the user to selectively supply energy to end effector assembly 100, e.g., upon actuation of activation button 92. As an alternative to actuation assembly 90 being disposed on distal body portion 22, a footswitch (not explicitly shown), or other activation device may be provided separate from forceps 10.

Continuing with reference to FIGS. 1 and 2, distal body portion 22 further defines a transverse cut-out 26 extending therethrough. Cut-out 26 extends through distal body portion 22 and is positioned so as to bifurcate lumen 24 into a proximal lumen section 27a and a distal lumen section 27b. A leaf spring 28 having a free proximal end 29a and a fixed distal end 29b is monolithically formed via the single-shot process that forms integral member 20 with and extends from distal body portion 22 into cut-out 26. More specifically, fixed distal end 29b of leaf spring 28 is formed with an inwardly-facing surface of distal body portion 22 that defines cut-out 26. Leaf spring 28 extends proximally from the inwardly-facing surface to free proximal end 29a, which is disposed within cut-out 26 and is biased towards a proximal end of cut-out 26. In this at-rest condition, leaf spring 28 defines an extended configuration. Leaf spring 28 is compressible, against its bias, from the extended configuration to a compressed configuration, wherein free proximal end 29a of leaf spring 28 is disposed adjacent fixed distal end 29b. Free proximal end 29a of leaf spring 28 include an engagement feature 29c, e.g., a saddle, configured to receive a portion of trigger assembly 70 so as to bias trigger 72 proximally, as detailed below.

Referring still to FIGS. 1 and 2, proximal handle portion 30 of integral member 20 includes two movable handles 30a, 30b disposed on opposite sides of distal body portion 22 and extending proximally from distal body portion 22. Handles 30a, 30b are integrally formed with distal body portion 22 via respective living hinges 31a, 31b. Handles 30a, 30b are formed during the same single-shot process used to form integral member 20. Living hinges 31a, 31b are configured so as to bias handles 30a, 30b towards a spaced-apart position relative to one another, although other configurations are also contemplated. Handles 30a, 30b are movable from this spaced-apart position to an approximated position to move jaw members 110, 120 of end effector assembly 100 (see FIG. 1) from an open position to a closed position for grasping tissue therebetween, as detailed below. Each handle 30a, 30b further includes a finger ring 32a, 32b defining a finger hole 33a, 33b, respectively, which facilitates the ability to grasp and manipulate handles 30a, 30b relative to one another.

Handles 30a, 30b of proximal handle portion 30 are integrally connected to one another via a connector member 40. Connector member 40 includes a first leg 41, a second leg 42, and a hub 43, all formed during the same single-shot process that forms integral member 20. First leg 41 is coupled to handle 30a via a living hinge 44, second leg 42 is coupled to handle 30b via a living hinge 45, and hub 43 is coupled between first and second legs 41, 42 via respective living hinges 46, 47. Hub 43 defines a lumen 48 extending therethrough that is disposed in coaxial alignment with lumen 24 of distal body portion 22. As a result of the above-detailed configuration of handles 30a, 30b and connector member 40, moving handles 30a, 30b from the spaced-apart position to the approximated position flexes living hinges 44, 45, 46, 47 and urges hub 43 to translate distally relative to e.g., towards, distal body portion 22. Return of movable handle 30a, 30b to the spaced-apart position, e.g., under the bias of living hinges 31a, 31b, on the other hand, permits living hinges 44, 45, 46, 47 to return under bias to their respective initial conditions, thereby urging hub 43 to translate proximally relative to, e.g., away from, distal body portion 22.

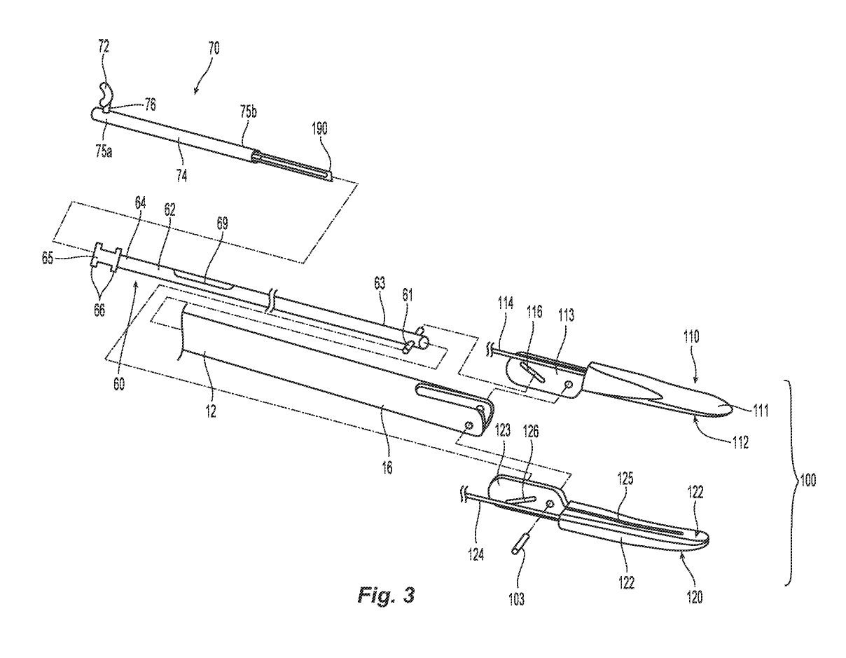

Referring to FIGS. 1 and 3, end effector assembly 100 is attached at distal end 16 of shaft 12 and includes a pair of opposing jaw members 110 and 120. Each jaw member 110, 120 includes an outer insulative jaw housing 111, 121, an electrically-conductive tissue-contacting surface 112, 122, and a proximal flange 113, 123, respectively. Tissue-contacting surfaces 112, 122 are disposed about jaw housings 111, 121, respectively, and include wires 114, 124, respectively, that extend through shaft 12, ultimately connecting tissue-contacting surfaces 112, 122 to activation button 92 (FIG. 1) and the source of energy (not shown), e.g., via the wires extending through cable 18 (FIG. 1), such that energy may be selectively supplied to tissue-contacting surface 112 and/or tissue-contacting surface 122 and conducted therebetween and through tissue grasped between jaw members 110, 120 to treat, e.g., cauterize, coagulate/desiccate, and/or seal, tissue. Further, any suitable energy modality may be used, e.g., electrosurgical, thermal, microwave, light, ultrasonic, etc., for energy-based tissue treatment.

Proximal flanges 113, 123 of jaw members 110, 120 are pivotably coupled to one another and shaft 12 via a pivot pin 103. End effector assembly 100 is designed as a bilateral assembly, i.e., where both jaw member 110 and jaw member 120 are movable about pivot 103 relative to one another and shaft 12. However, end effector assembly 100 may alternatively be configured as a unilateral assembly, i.e., where one of the jaw members 110, 120 is fixed relative to shaft 12 and the other jaw member 110, 120 is movable about pivot 103 relative to shaft 12 and the fixed jaw member 110, 120. Proximal flanges 113, 123 of jaw members 110, 120, respectively, each further include an oppositely-angled cam slot 116, 126 defined therethrough that is configured to receive a drive pin 61. Drive pin 61 is mounted at distal end 63 of drive bar 62 of drive assembly 60 such that, as will be described in greater detail below, reciprocation of drive bar 62 through shaft 12 and relative to end effector assembly 100 effects pivoting of jaw members 110, 120 relative to one another between the open and closed positions. More specifically, cam slots 116, 126 are oriented such that distal translation of drive pin 61 effects pivoting of jaw members 110, 120 from the open position to the closed position and such that proximal translation of drive pin 61 effects pivoting of jaw members 110, 120 from the closed position to the open position.

A knife channel 115 extends longitudinally through one (or both) jaw members 110, 120, e.g., jaw member 120, to facilitate reciprocation of knife 190 between jaw members 110, 120 to cut tissue disposed therebetween, e.g., upon actuation of trigger 72 of trigger assembly 70. That is, knife 190 is operatively coupled to trigger assembly 70 such that actuation of trigger 72 advances knife 190 from a retracted position, wherein knife 190 is positioned proximally of jaw members 110, 120, to a deployed position, wherein knife 190 extends between jaw members 110, 120 and through channel 115 to cut tissue grasped between jaw members 110, 120. Knife 190 may be configured for mechanical cutting (as shown), or may be energizable, e.g., electrically coupled to the source of energy (not shown) via one or more wires (not shown), for electromechanically cutting tissue. Trigger assembly 70 is described in greater detail below.

With reference to FIGS. 1-3, drive assembly 60, as mentioned above, includes a drive bar 62 having a drive pin 61 mounted at distal end 63 of drive bar 62 to pivot jaw members 110, 120 between the open and closed positions in response to translation of drive bar 62 through and relative to shaft 12. Drive bar 62 is slidably disposed within shaft 12 and extends proximally from shaft 12 into and through integral member 20. More specifically, drive bar 62 extends proximally from shaft 12, through proximal lumen section 27a of lumen 24, cut-out 26, and distal lumen section 27b of lumen 24, exiting proximal end 23a of distal body portion 22 of integral member 20. A mandrel 65 is disposed about proximal end 64 of drive bar 62, proximally of distal body portion 22 of integral member 20. Mandrel 65 includes a pair of spaced-apart annular flanges 66 and is engaged within lumen 48 of hub 43 of connector member 40 with one of the flanges 66 disposed on either end of hub 43 such that mandrel 65 and, thus, drive bar 62 are fixedly coupled to connector member 40.

As a result of the above-detailed configuration, moving handles 30a, 30b from the spaced-apart position to the approximated position flexes living hinges 44, 45, 46, 47 and urges hub 43 to translate distally relative to, e.g., towards, distal body portion 22 such that mandrel 65 and drive bar 62 are likewise translated distally to pivot jaw members 110, 120 from the open position to the closed position. Return of movable handles 30a, 30b to the spaced-apart position, e.g., under the bias of living hinges 31a, 31b, on the other hand, permits living hinges 44, 45, 46, 47 to return under bias to their respective initial conditions, thereby urging hub 43 to translate proximally relative to, e.g., away from, distal body portion 22 such that mandrel 65 and drive bar 62 are likewise translated proximally to pivot jaw members 110, 120 from the closed position back to the open position. The bias of living hinges 31a, 31b, as noted above, biases handles 30a, 30b towards the spaced-apart position, thereby biasing jaw members 110, 120 towards the open position, although other configurations are also contemplated.

Continuing with reference to FIGS. 1-3, trigger assembly 70 includes trigger 72, a knife drive bar 74, and a post 76. Knife drive bar 74 is slidably disposed within cut-out 26, distal lumen section 27b of lumen 24, and drive bar 62, and defines a proximal end 75a and a distal end 75b. Knife 190 is coupled to and extends distally from distal end 75b of knife drive bar 74. Post 76 is situated within cut-out 26 towards proximal end 75a of knife drive bar 74 and extends transversely from knife drive bar 74 through a slot 69 defined within drive bar 62 to fixedly interconnect the externally-disposed trigger 72 with knife drive bar 74.

Post 76 of trigger assembly 70 is operably engaged with free proximal end 29a of leaf spring 28, e.g., a portion of post 76 is at least partially received within saddle 29c of leaf spring 28, such that leaf spring 28 biases post 76 proximally relative to cut-out 26 and distal body portion 22 of integral member 20. Trigger 72, which extends from cut-out 26 to facilitate grasping and manipulation by a user, is slidable along cut-out 26 and relative to distal body portion 22 between a proximal position, wherein post 76 is positioned adjacent the proximal end of cut-out 26, and a distal position, wherein post 76 is positioned adjacent the distal end of cut-out 26. In the proximal position of trigger 72, leaf spring 28 is extended. In the distal position of trigger 72, on the other hand, leaf spring 28 is compressed.

As can be appreciated in view of the above-detailed configuration, the proximal position of trigger 72 corresponds to the retracted position of knife 190, and the distal position of trigger 72 corresponds to the extended position of knife 190. Thus, distal sliding of trigger 72 along cut-out 26 from the proximal position to the distal position extends knife 190 between jaw members 110, 120 to cut tissue grasped therebetween. Leaf spring 28 functions to bias post 76 and, thus, knife drive bar 74 proximally, thus biasing knife 190 towards the retracted position, although other configurations are also contemplated.

Referring still to FIGS. 1-3, in use, with jaw members 110, 120 disposed in the open position, forceps 10 is initially maneuvered into position such that tissue to be treated is disposed between jaw members 110, 120 of end effector assembly 100. Once the desired position has been achieved, handles 30a, 30b are moved from the spaced-apart position to the approximated position such that hub 43 is urged distally, thereby translating drive bar 62 distally through integral member 20 and shaft 12 to pivot jaw members 110, 120 to the closed position to grasp tissue between tissue-contacting surfaces 112, 122, respectively.

With tissue grasped between tissue-contacting surfaces 112, 122, energy may be supplied to tissue-contacting surfaces 112, 122 and conducted through tissue to treat tissue via actuation of activation button 92. Additionally or as an alternative to tissue treatment, depending on a particular purposes, trigger 72 may be slid distally to translate knife drive bar 76 distally though drive bar 62 and relative to end effector assembly 100 to advance knife 190 between jaw members 110, 120 to cut tissue grasped therebetween. Thereafter, knife 190 is returned to the retracted position, e.g., via releasing trigger 72 to allow leaf spring 28 to return trigger 72 to the proximal position under its bias, and jaw members 110, 120 are moved back to the open position, e.g., via releasing handles 30a, 30b to allow living hinges 31a, 31b to return handles 30a, 30b to the spaced-apart position under their bias.

With respect to the manufacture of forceps 10, as mentioned above, integral member 20 is formed via a single-shot process, e.g., a single-shot overmold or single-shot injection mold, although other suitable single-shot processes are also contemplated. Forming integral member 20 in a single-shot is advantageous in that in eliminates the need for complex parts and/or manufacturing steps for assembling the various components of a forceps, e.g., the body or housing portions, handle assembly, biasing mechanisms, etc. As noted above, in embodiments, shaft 12 may be integrally formed with integral member 20 via the single-shot molding, thus further reducing the components that require assembly. In embodiments where shaft 12 is not integrally formed, lumen 24 is formed during the single-shot process to readily enable coupling of shaft 12 therein. In either configuration, providing a single-shot integral member simplifies manufacturing and reduces cost.

Once integral member 20 has been formed, prior thereto, or concurrently therewith, the various other operable components of forceps 10 are coupled to one another and integral member 20 to fully assemble forceps 10. Although one order of assembly is detailed for exemplary purposes below, it is envisioned that the various operable components of forceps 10 be coupled to one another and integral member 20 in any suitable order and/or in any suitable fashion.

Insertion of pin 103 through shaft 12 and the proximal flanges 113, 123 of jaw members 110, 120 may be effected to operably couple end effector assembly 100 to shaft 12. Thereafter or prior thereto, pin 61 of drive assembly 60 may be inserted into slots 116, 126 defined within proximal flanges 113, 123 of jaw members 110, 120 to operably couple drive assembly 60 to end effector assembly 100. Shaft 12 may then be inserted into lumen 24 of distal body portion 22 of integral member 20 and secured therein in any suitable fashion (in embodiments where shaft 12 is not integrally formed with integral member 20). Drive assembly 60 is coupled to hub 43 of connector member 40 via the engagement of mandrel 65 within lumen 48 of hub 43. Prior to or after the above, trigger assembly 70 and knife 190 are slidably disposed within drive assembly 60 and operably coupled to integral member 20, e.g., via engagement of saddle 29c of free end 29b of leaf spring 28 with post 76 of trigger assembly 70.

Activation button 92 may, thereafter or prior thereto, be coupled within an activation button housing 93 defined within distal body portion 22 of integral member 20. Activation button housing 93 may be formed within distal body portion 22 via the single-shot process. Activation button 92 is also electrically connected to end effector assembly 100 and cable 18, e.g., via the one or more wires extending through distal body portion 22, cable 18, and/or shaft 12.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A forceps, comprising:
an integral member, including:
a body portion defining a lumen extending longitudinally therethrough;
first and second handles integrally formed with the body portion via living hinges, the first and second handles movable relative to one another between a spaced-apart position and an approximated position; and
a connector member integrally formed between the first and second handles, the connector member including a first leg coupled to the first handle via a living hinge, a second leg coupled to the second handle via a living hinge, and a hub coupled between the first and second legs via living hinges, wherein movement of the first and second handles between the spaced-apart position and the approximated position urges the hub to translate relative to the body portion,
wherein the integral member is adapted to receive a shaft that supports an end effector assembly for treating tissue.

2. The forceps according to claim 1, wherein the hub of the integral member is configured to engage a drive assembly disposed within the shaft such that movement of the first and second handles to the approximated position urges the hub to translate the drive assembly to actuate the end effector assembly.

3. The forceps according to claim 2, wherein the end effector assembly includes first and second jaw members movable from an open position to a closed position for grasping tissue and wherein the drive assembly includes a drive bar, wherein movement of the first and second handles from the spaced-apart position to the approximated position urges the hub to translate distally relative to the body portion, thereby translating the drive bar of the drive assembly distally relative to the end effector assembly to move the first and second jaw members from the open position to the closed position.

4. The forceps according to claim 3, wherein the drive assembly includes a mandrel disposed about the proximal end of the drive bar, the mandrel engaged within a lumen defined through the hub.

5. The forceps according to claim 3, wherein at least one of the jaw members is adapted to connect to a source of energy for conducting energy between the jaw members to treat tissue grasped therebetween.

6. The forceps according to claim 3, further including a knife slidably disposed within the drive bar, the knife movable between a retracted position, wherein the knife is positioned proximally of the end effector assembly, and an extended position, wherein the knife extends at least partially between the jaw members to cut tissue grasped therebetween.

7. The forceps according to claim 6, further including a trigger assembly including a trigger and a trigger drive bar defining a proximal end and a distal end, the knife engaged to the distal end of trigger drive bar, the trigger coupled to the proximal end of the trigger drive bar, the trigger movable between a proximal position and a distal position for moving the knife between the retracted position and the extended position.

8. The forceps according to claim 7, wherein the trigger is positioned adjacent a cut-out defined within the body portion of the integral member, and wherein the trigger drive bar extends through the drive bar of the drive assembly.

9. The forceps according to claim 8, wherein the integral member includes a leaf spring integrally formed therewith and extending into the cut-out, the leaf spring positioned to bias the trigger proximally, thereby biasing the knife towards the retracted position.

10. The forceps according to claim 9, wherein the trigger assembly includes a post interconnecting the trigger and the trigger drive bar, and wherein the leaf spring includes an engagement member disposed at a free end thereof, the engagement member configured to at least partially receive the post in operable engagement therewith to bias the trigger proximally.

11. A forceps, comprising:
an integral member, including:
a body portion defining a lumen extending longitudinally therethrough;
first and second handles integrally formed with the body portion via living hinges, the first and second handles movable relative to one another between a spaced-apart position and an approximated position;
a connector member integrally formed between the first and second handles, the connector member including a first leg coupled to the first handle via a living hinge, a second leg coupled to the second handle via a living hinge, and a hub coupled between the first and second legs via living hinges, wherein movement of the first and second handles between the spaced-apart position and the approximated position urges the hub to translate relative to the body portion; and
a leaf spring integrally formed with the body portion,
wherein the integral member is adapted to receive a shaft that supports an end effector assembly for treating tissue, and
wherein the leaf spring is adapted to bias a trigger assembly for actuating a knife relative to the end effector assembly.

12. The forceps according to claim 11, wherein the hub of the integral member is configured to engage a drive assembly disposed within the shaft such that movement of the first and second handles to the approximated position urges the hub to translate the drive assembly to actuate the end effector assembly.

13. The forceps according to claim 12, wherein the end effector assembly includes first and second jaw members movable from an open position to a closed position for grasping tissue and wherein the drive assembly includes a drive bar, wherein movement of the first and second handles from the spaced-apart position to the approximated position urges the hub to translate distally relative to the body portion, thereby translating the drive bar of the drive assembly distally relative to the end effector assembly to move the first and second jaw members from the open position to the closed position.

14. The forceps according to claim 13, wherein the drive assembly includes a mandrel disposed about the proximal end of the drive bar, the mandrel engaged within a lumen defined through the hub.

15. The forceps according to claim 13, wherein at least one of the jaw members is adapted to connect to a source of energy for conducting energy between the jaw members to treat tissue grasped therebetween.

16. The forceps according to claim 13, wherein the knife is slidably disposed within the drive bar, the knife movable between a retracted position, wherein the knife is positioned proximally of the end effector assembly, and an extended position, wherein the knife extends at least partially between the jaw members to cut tissue grasped therebetween.

17. The forceps according to claim 16, wherein the trigger assembly includes a trigger and a trigger drive bar defining a proximal end and a distal end, the knife engaged to the distal end of trigger drive bar, the trigger coupled to the proximal end of the trigger drive bar, the trigger movable between a proximal position and a distal position for moving the knife between the retracted position and the extended position.

18. The forceps according to claim 17, wherein the trigger is positioned adjacent a cut-out defined within the body portion of the integral member, and wherein the trigger drive bar extends through the drive bar of the drive assembly.

19. The forceps according to claim 18, wherein the leaf spring extends into the cut-out, the leaf spring positioned to bias the trigger proximally, thereby biasing the knife towards the retracted position.

20. The forceps according to claim 19, wherein the trigger assembly includes a post interconnecting the trigger and the trigger drive bar, and wherein the leaf spring includes an engagement member disposed at a free end thereof, the engagement member configured to at least partially receive the post in operable engagement therewith to bias the trigger proximally.
